(19) United States
(12) Patent Application Publication
(10) Pub. No.: US 2023/0076818 A1
(43) Pub. Date: Mar. 9, 2023

(54) INHIBITION OF CORONAVIRUS (COVID-19) INFECTION BY MEANS OF APIVIRINE EXTRACT - DICHROSTACHYS GLOMERATA

(71) Applicant: Achidi Valentin AGON, Cotonou (BJ)

(72) Inventors: Achidi Valentin AGON, Cotonou (BJ); Celestin KINNOUDO, Cotonou (BJ)

(21) Appl. No.: 17/923,621

(22) PCT Filed: May 7, 2020
(86) PCT No.: PCT/IB2020/054304

§ 371 Date: Nov. 7, 2022

(51) Int. Cl. A61K 36/48; A61P 31/14

(52) U.S. Cl. CPC A61K 36/48; A61P 31/14

(57) ABSTRACT

APIVIRINE, extracted from Dichrostachys glomerata, is an antiviral drug made up of: gallic tannins, catechic tannins, alkaloids, flavonoids, saponosides, anthocyanins, leucoanthocyanins, steroids and terpenes, which together exhibit anti-COVID-19 activity. It can be used to cure and prevent coronavirus disease in symptomatic and asymptomatic patients and all exposed persons. Toxicological tests on rats are conclusive: APIVIRINE is non-toxic. The starting dose in cases requiring respiratory assistance is 12 capsules each containing 350 mg (orally or through a tube) of APIVIRINE powder obtained by incorporating 500 mg of pure lyophilized extract. Said dose is given three times a day in accordance with the half-life of APIVIRINE (four horns). For less severe cases and asymptomatic cases, the dose is three capsules three times a day and, for prevention, the dosage is one to two capsules three times a day. The absorption of APIVIRINE leads the coronavirus disappearing.

### TECHNICAL FIELD: BIOPHARMACY

The extraction of Dichrostachys glomerata is used to manufacture an antiviral medication with immediate effectiveness on COVID-19. The treatment offered by this medication consists in neutralizing the COVID-19 to be verified by PCR and as a consequence the patient recovers their perfect health. The natural origin of this active ingredient makes it a medication that escapes the possibility of development of resistance to CORONAVIRUS COVID-19.

The technique used involves the extraction of the total extract followed by its purification in order to qualitatively and quantitatively extract the chemical families present in the densified total extract. This is the approach we used previously in 2002 in our application PCT/IB02/05285, filing date: Dec. 11, 2002, international publication No. WO2004/052384 A1, of Jun. 24, 2004. The substance is chemically processed to provide highly purified and concentrated molecular families for fast and efficient activity. This active ingredient, previously named APIVIRINE, is now in its most refined form with a very low incorporation rate and a spectacular anti-viral activity.

### PRIOR ART

Starting from the therapeutic virtues of a plant, the processes and techniques used for the extraction of the active principle or active ingredient vary according to the objectives. For example, we can extract essential oils from a plant by distillation, it is possible to use water or alcohol or an oil to extract active principles depending on whether the latter is water-soluble or soluble in alcohol or liposoluble.

### 1—THE PLANT

1-1—Scientific name: Dichrostachys glomerate

(in the local language FON (national language of BENIN the name is)

1-2- BADAWIN

1-3—Name in English: bell mimosa

1-4—Botanical family: mimosaceae

1-5—Botanical source: tropical plant distributed in all of West Africa.

1-6—The substance extracted from the plant is acidic

1-7—Chemotype: 0 to 500 meters of altitude in tropical region

(Altitude at which the plant is very rich in this therapeutic property and non-toxic according to our research sites from the south to the north of BENIN)

1-8—Maturity: The adult plant is useful at all times, only the young shoots of the beginning of the rainy seasons must be harvested at least two months after the first rains of the year.

1-9—Useful part of the plant: The shoot of the plant (the crown or foliage) except for branches with diameters greater than two centimeters.

NB: The natural ratio of quantity of leaves/branch during the rainy season must be respected (the plant is very dense in rainy season and the opposite applies during drought)

1-10—Description of the plant: plant characterized by very pronounced strange spines, its leaves are very small, its flowers attract bees, it is a leguminous plant which produces pods folded upon itself.

1-11—Large scale reproducibility: This plant is easily reproducible on a large scale, at the beginning of the rainy seasons, the young green shoots that emerge from the roots are very numerous and are easy to transplant.

1-12—TOXICITY: this medicinal plant is not toxic in any way decoction, expression, infusion, maceration, kneading, pounding, pulverization, softening, roasting and oil crushing.

### 2—PHYTOCHEMICAL SCREENING

The phytochemical screening of the DICHROSTACHYS GLOMERATA extracts revealed the following chemical components with their dosage: two chemical families are predominantly present (+++): gallic tannins and catechic tannins; three are abundant (++): alkaloids, flavonoids and saponosides; four are less abundant (+): anthocyanins, leucoanthocyanins, steroids and terpenes and in trace form (+−): essential oils. Others: quinone derivatives, cardenolides, cyanogenetic derivatives and mucilages are absent.

### 3—TOXICOLOGICAL STUDIES OF THE EXTRACTS

Toxicity studies are conducted on rats to assess the toxicity of the plant extracts.

### 3-1—Study of Toxicity in Rats

The studies are conducted at a temperature of 25° C.
Ten (10) Wistar rats weighing an average of 227 grams each receive a 2.5 ml dose of the test substance orally twice daily. The animals are then observed for toxicological activity of the Dichrostachys glomerate extract at 1, 3, 6 and 24 hours after treatment and once a day thereafter for 14 days. All the animals have survived the observation period. Furthermore, no discomfort or morbid effects were observed during the period.

| | | Male | Female | Dose | Morbid | Mortality |
|---|---|---|---|---|---|---|
| | Race | gender | gender | administered | effects | (%) |
| Rats | Wistar | 05 | 05 | 22 ml/kg | None | 0 |

In sub-chronic toxicity studies in rats, the Dichrostachys glomerata extract named Apivirine did not show any toxic effect, even after an exposure well above the human exposure. Apivirine therefore does not present a risk in humans for the indications for which it is intended.

### 4—MICROBIOLOGICAL STUDIES

Microbiological studies are conducted on the extracts of the plant

| | | | | | SULFITE- | | |
|---|---|---|---|---|---|---|---|
| SEARCH | TOTAL | COLIMETRY CFU/G | | STAPHYLOCOCCUS | REDUCING | YEASTS | |
| CARRIED | FLORA | TOTAL | FECAL | AUREUS | ANAEROBES | MOLD | SALMONELLA |
| OUT | CFU/G | COLIFORMS | COLIFORMS | CFU/G | CFU/G | CFU/G | in 25 G |
| METHODS | V.08-051 | V.08-050 | V.08-060-1 | V.08-057-1 | XPV.08-061 | XPV 08-059 | NFV.08-059 |
| RESULTS | <1 | <1 | <1 | <1 | <1 | L < 1 | None |
| | | | | | | M < 1 | |
| m | | | | | | | |
| STANDARDS* | | | | | | | |
| M | | | | | | | |

Results: Absence of germs

### 5—IN VIVO STUDY OF DICHROSTACHYS GLOMERATA EXTRACTS, NAMED APIVIRINE, DOSED AT 350 MG ON PATIENTS SUFFERING FROM CORONAVIRUS

Twenty patients suffering from coronavirus voluntarily took the extracts of Dichrostachys glomerate, named APIVIRINE, dosed at 350 mg in blister capsules. The results of 16 people who have accepted that their name be coded with their testimony relating to this therapy and be made public, are presented as follows:

### 5-1 COVID-19 Pharmacotherapeutic Protocol

Symptoms of COVID-19 disease range from irritation of the respiratory tract to true respiratory distress with persistent fever. This notorious ailment, which is characterized by inflammation of the airways, convulsions, fevers, etc., subsides within the 1^{st} and 2^{nd} day of taking the recommended dose of APIVIRINE. Indeed, in most patients, the oral administration of the first twelve capsules results in the modification of the most characteristic symptoms of the coronavirus viral infection: respiratory problems, generalized asthenia which is proof of an increased viral replication, loss of taste and smell, etc. As a result, cephalic ailments accompanied by weakness disappear making way for generalized muscular stamina and physical and emotional energy whose comforting immediacy amazes both the patient and those treating them. Some of the patients diagnosed with tingling, itching and congestion of their throat regain a normal and relaxed feeling of the throat in a record time of only 3 days of treatment. It is moreover noted that the disturbing and debilitating diarrhea in some patients subsided after two days of treatment with APIVIRINE. Signs of dizziness and cloudiness noted in some patients prior to administration of APIVIRINE improved after the use of about one package of the product.

Another very striking phenomenon was the very rapid disappearance of the whole sickly state which was manifested by the acceleration of the pulse and the increase of the fever in all the patients. Asymptomatic persons who tested positive for COVID-19 tested negative after 4 days of treatment with a single package of APIVIRINE. People suffering from other chronic diseases such as arterial hypertension, diabetes, renal failure, hepatitis B, sickle cell disease, etc., who are potentially at risk, generally heal from COVID-19 after using at least one package of APIVIRINE.

The team of researchers and caregivers who look after patients suffering from COVID-19 in Burkina Faso is constantly aware of the risk of being infected. This risk is greater because of the ease and speed of contagion in the context of the COVID-19 pandemic.

In fact, as a preventive measure, one or two capsules taken 3 times a day were enough to ensure that no member of this team was infected with the coronavirus while we were in contact with infected persons. For example, upon the return home of the Beninese team on Tuesday, Mar. 31, 2020, the national rules protocol requires that anyone returning from an infected country must be quarantined for two weeks. At the end of this period, which is equivalent to the incubation period of COVID-19, the tests (from observation of possible clinical signs, to rapid tests, to PCR tests, etc.) for coronavirus were carried out and all proved to be negative. None of the team members is therefore infected with coronavirus.

In conclusion, APIVIRINE is the ideal anti-viral drug for the treatment and prevention of COVID-19 disease in symptomatic and asymptomatic patients and in all patients at risk of infection. Moreover, the case of COVID-19 sufferers with other chronic diseases, such as hypertension, diabetes, etc., does not worsen. Toxicology tests carried out on rats in 2007 at the National Laboratory of Toxicology and Narcotics in Cotonou, Benin and in 2020 at the Institute of Research in Health Sciences in Ouagadougou, Burkina Faso were conclusive: APIVIRINE is not a toxic substance for humans.

Finally, in the most serious cases requiring respiratory assistance, the loading dosage is the daily administration by oral route (or by direct feeding in the stomach by nasogastric tube) of 12 capsules each containing 350 mg of APIVIRINE powder resulting from the incorporation of 500 mg of pure lyophilized extract. This loading dosage must be divided 3 times during the day because of the plasma life span (half-life) of APIVIRINE which is at least 4 hours. Thus, for moderately severe and asymptomatic cases, the dosage is 3 capsules 3 times a day and for prevention, the dosage is 1 to 2 capsules 3 times a day. Upon passage of APIVIRINE, the coronavirus passes away.

Patient No. 1

a) Age: 58

b) Gender: M

c) Date of first symptoms: Mar. 6, 2020

d) Symptoms: cough, headache, asthenia

e) Date of diagnosis of COVID-19: Mar. 11, 2020

f) APIVIRINE treatment start date: Mar. 11, 2020

g) End date of the symptoms: Mar. 20, 2020

h) Date of the COVID-19 negative control: Mar. 25, 2020

i) Date APIVIRINE treatment stopped: Mar. 28, 2020

j) Number of APIVIRINE packages used: 05

k) Other chronic diseases: None

Patient No. 2

a) Age: 62

b) Gender: F

c) Date of first symptoms: Mar. 4, 2020

d) Symptoms: cough, headache, asthenia

e) Date of diagnosis of COVID-19: Mar. 11, 2020

f) APIVIRINE treatment start date: Mar. 11, 2020

g) End date of the symptoms: Mar. 13, 2020

h) Date of the COVID-19 negative control: Mar. 19, 2020

i) Date APIVIRINE treatment stopped: Mar. 23, 2020

j) Number of APIVIRINE packages used: 04

k) Other chronic diseases: sickle cell disease

Patient No. 3

a) Age: 27

b) Gender: M

c) Date of first symptoms: Mar. 10, 2020

d) Symptoms: cough

e) Date of diagnosis of COVID-19: Mar. 16, 2020

f) APIVIRINE treatment start date: Mar. 16, 2020

g) End date of the symptoms: Mar. 20, 2020

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 4

a) Age: 25

b) Gender: F

c) Date of first symptoms: Mar. 5, 2020

d) Symptoms: cough

f) Date of diagnosis of COVID-19: Mar. 16, 2020

g) APIVIRINE treatment start date: Mar. 16, 2020

h) End date of the symptoms: Mar. 20, 2020

i) Date of the COVID-19 negative control: Mar. 29, 2020

j) Date APIVIRINE treatment stopped: Mar. 19, 2020

k) Number of APIVIRINE packages used: 01

l) Other chronic diseases: None

Patient No. 5

a) Age: 19

b) Gender: F

c) Date of first symptoms: Mar. 6, 2020

d) Symptoms: cough

e) Date of diagnosis of COVID-19: Mar. 16, 2020

f) APIVIRINE treatment start date: Mar. 16, 2020

g) End date of the symptoms: Mar. 20, 2020

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 6

a) Age: 06

b) Gender: F

c) Date of first symptoms: —

d) Symptoms: none

e) Date of diagnosis of COVID-19: Mar. 16, 2020

f) APIVIRINE treatment start date: Mar. 16, 2020

g) End date of the symptoms: —

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 7

a) Age: 48

b) Gender: F

c) Date of first symptoms: —

d) Symptoms: throat irritation, diarrhea

e) Date of diagnosis of COVID-19: Mar. 31, 2020

f) APIVIRINE treatment start date: Mar. 31, 2020

g) End date of the symptoms: —

h) Date of the COVID-19 negative control: Apr. 10, 2020

i) Date APIVIRINE treatment stopped: ongoing

j) Number of APIVIRINE packages used: 04

k) Other chronic diseases: None

Patient No. 8

a) Age: 20

b) Gender: F

c) Date of first symptoms: —

d) Symptoms: vertigo, vomiting, fever, palpitations

e) Date of diagnosis of COVID-19: Mar. 30, 2020

f) APIVIRINE treatment start date: Mar. 30, 2020

g) End date of the symptoms: —

h) Date of the COVID-19 negative control: Apr. 10, 2020

i) Date APIVIRINE treatment stopped: ongoing

j) Number of APIVIRINE packages used: 04

k) Other chronic diseases: None

Patient No. 9

a) Age: 58

b) Gender: M

c) Date of first symptoms: Mar. 6, 2020

d) Symptoms: cough, headache, asthenia

e) Date of diagnosis of COVID-19: Mar. 11, 2020

f) APIVIRINE treatment start date: Mar. 11, 2020

g) End date of the symptoms: Mar. 20, 2020

h) Date of the COVID-19 negative control: Mar. 25, 2020

i) Date APIVIRINE treatment stopped: Mar. 28, 2020

j) Number of APIVIRINE packages used: 05

k) Other chronic diseases: None

Patient No. 10

a) Age: 62

b) Gender: F

c) Date of first symptoms: Mar. 4, 2020

d) Symptoms: cough, headache, asthenia

e) Date of diagnosis of COVID-19: Mar. 11, 2020

f) APIVIRINE treatment start date: Mar. 11, 2020

g) End date of the symptoms: Mar. 13, 2020

h) Date of the COVID-19 negative control: Mar. 19, 2020

i) Date APIVIRINE treatment stopped: Mar. 23, 2020

j) Number of APIVIRINE packages used: 04

k) Other chronic diseases: Sickle cell disease

Patient No. 11

a) Age: 27

b) Gender: M

c) Date of first symptoms: Mar. 10, 2020

d) Symptoms: cough

e) Date of diagnosis of COVID-19: Mar. 16, 2020

f) APIVIRINE treatment start date: Mar. 16, 2020

g) End date of the symptoms: Mar. 20, 2020

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 12

a) Age: 25

b) Gender: F

c) Date of first symptoms: Mar. 5, 2020

d) Symptoms: cough

e) Date of diagnosis of COVID-19: Mar. 16, 2020

f) APIVIRINE treatment start date: Mar. 16, 2020

g) End date of the symptoms: Mar. 20, 2020

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 13

a) Age: 19

b) Gender: F

c) Date of first symptoms: Mar. 6, 2020

d) Symptoms: cough

e) Date of diagnosis of COVID-19: Mar. 16, 2020

f) APIVIRINE treatment start date: Mar. 16, 2020

g) End date of the symptoms: Mar. 20, 2020

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 14

a) Age: 06

b) Gender: F

c) Date of first symptoms: not noted—

d) Symptoms: none

e) Date of diagnosis of COVID-19: Mar. 16, 2020

f) APIVIRINE treatment start date: Mar. 16, 2020

g) End date of the symptoms: Mar. 19, 2020

h) Date of the COVID-19 negative control: Mar. 29, 2020

i) Date APIVIRINE treatment stopped: Mar. 19, 2020

j) Number of APIVIRINE packages used: 01

k) Other chronic diseases: None

Patient No. 15

a) Age: 58

b) Gender: M

c) Date of first symptoms: Mar. 12, 2020

d) Symptoms: sore throat+fever

e) Date of diagnosis of COVID-19: Mar. 23, 2020

f) APIVIRINE treatment start date: Mar. 22, 2020

g) End date of the symptoms: Mar. 30, 2020

h) Date of the COVID-19 negative control: Apr. 4, 2020

i) Date APIVIRINE treatment stopped: Apr. 4, 2020

j) Number of APIVIRINE packages used: 04

k) Other chronic diseases: Hepatitis B

Patient No. 16

a) Age: 62

b) Gender: M

c) Date of first symptoms: Apr. 6, 2020

d) Symptoms: cough and fatigue

e) Date of diagnosis of COVID-19: Apr. 9, 2020

f) APIVIRINE treatment start date: Apr. 11, 2020

g) End date of the symptoms: Apr. 12, 2020

h) Date of the COVID-19 negative control: Apr. 14, 2020

i) Date APIVIRINE treatment stopped: Apr. 14, 2020

j) Number of APIVIRINE packages used: 01

k) Other chronic diseases: hypertension

1. Use of organic or synthetic extracts of the medicinal plant with the scientific name Dichrostachys glomerata for the manufacture of an antiviral medicine intended for the treatment of infection caused by the coronavirus COVID-19
